(19) United States
(12) Patent Application Publication
(10) Pub. No.: US 2017/0049774 A1
(43) Pub. Date: Feb. 23, 2017

(54) SUSTAINED RELEASE ORAL PHARMACEUTICAL COMPOSITIONS OF TOFACITINIB

(71) Applicant: SUN PHARMACEUTICAL INDUSTRIES LIMITED, Mumbai (IN)

(72) Inventors: Romi Barat SINGH, Varanasi (IN); Kalaiselvan RAMARAJU, Trichirapalli (IN); Sandeep Kumar VATS, Sonipat City (IN); Pulak Kumar METIA, Howrah (IN); Balamurali Krishna NOOKA, Srikakulam (IN)

(21) Appl. No.: 15/233,346

(22) Filed: Aug. 10, 2016

(30) Foreign Application Priority Data
Aug. 19, 2015 (IN) 2560/DEL/2015
Dec. 17, 2015 (IN) 4146/DEL/2015

(51) Int. Cl. A61K 31/519; A61K 9/28; A61K 9/20; A61K 9/00

(52) U.S. Cl. CPC A61K 31/519; A61K 9/0053; A61K 9/2886; A61K 9/2086; A61K 9/2866; A61K 9/2853; A61K 9/2846; A61K 9/282; A61K 9/2813; A61K 9/2027; A61K 9/2054; A61K 9/2018; A61K 9/2013; A61K 9/0004

(57) ABSTRACT

The present invention relates to sustained release oral pharmaceutical compositions of tofacitinib comprising tofacitinib, a release controlling polymer, and pharmaceutically acceptable excipients, wherein the sustained release oral pharmaceutical compositions further comprise an outer modified release coating that includes a modified release polymer. The invention also relates to a process for the preparation of the compositions.

### FIELD OF THE INVENTION

The present invention relates to sustained release oral pharmaceutical compositions of tofacitinib comprising tofacitinib, a release controlling polymer, and pharmaceutically acceptable excipients, wherein the sustained release oral pharmaceutical compositions further comprise an outer modified release coating that includes a modified release polymer. The invention also relates to a process for the preparation of the compositions.

### BACKGROUND OF THE INVENTION

Tofacitinib citrate is a Janus kinase inhibitor, which is chemically designated as (3R,4R)-4-methyl-3-(methyl-7H-pyrrolo[2,3-d]pyrimidin-4-ylamino)-β-oxo-1-piperidinepropanenitrile, 2-hydroxy-1,2,3-propanetricarboxylate (1:1).

Processes for the preparation of tofacitinib are disclosed in U.S. Pat. Nos. RE41,783 and 7,301,023. A process for the preparation of tofacitinib citrate is disclosed in U.S. Pat. No. 6,965,027.

U.S. Publication No. 2013/0344149 discloses oral dosage forms comprising tofacitinib suitable for modified release.

Oral sustained release formulations of tofacitinib are also disclosed in U.S. Publication No. 2014/0271842. This application discloses once-daily oral formulations of tofacitinib with a shorter duration of release. The claimed pharmaceutical dosage forms of tofacitinib disclosed therein release more than 75% of the drug in 5 hours.

The present invention discloses alternate sustained release pharmaceutical compositions of tofacitinib.

### SUMMARY OF THE INVENTION

The present invention relates to sustained release oral pharmaceutical compositions of tofacitinib comprising tofacitinib, a release controlling polymer, and pharmaceutically acceptable excipients, wherein the sustained release oral pharmaceutical compositions further comprise an outer modified release coating that includes a modified release polymer. The invention also relates to a process for the preparation of the compositions.

### DETAILED DESCRIPTION OF THE INVENTION

A first aspect of the present invention provides a sustained release oral pharmaceutical composition of tofacitinib comprising tofacitinib, a release controlling polymer, and pharmaceutically acceptable excipients, wherein the sustained release oral pharmaceutical composition further comprises an outer modified release coating.

According to a first embodiment of the above aspect, the composition comprises a core comprising tofacitinib, optionally a first coating over the core, and an outer modified release coating either over the core or over the first coating.

According to a first embodiment of the above aspect, the first coating comprises from about 5% by weight to about 20% by weight of the core weight, and the outer coating comprises from about 1% by weight to about 15% by weight either of the core weight or weight of the coated core coated with the first coating.

According to a second embodiment of the above aspect, the composition further comprises an acidifying agent or a surfactant or combinations thereof.

According to a third embodiment of the above aspect, the composition has an in-vitro release profile such that the pharmaceutical composition releases not more than 30% of the tofacitinib in 1 hour, not less than 35% and not more than 75% of tofacitinib in 2.5 hours and not less than 75% of tofacitinib in 5 hours.

According to a fourth embodiment of the above aspect, the composition has a release profile such that it releases less than 35% of the tofacitinib in 2.5 hours.

According to a fifth embodiment of the above aspect, the composition has a release profile such that it releases less than 75% of the tofacitinib in 5 hours.

According to a sixth embodiment of the above aspect, the composition comprises tofacitinib having a particle size distribution D₉₀ value of about 30 μm or less, D₅₀ value of about 20 μm or less, and D₁₀ value of about 5 μm or less.

According to a seventh embodiment of the above aspect, the composition comprises tofacitinib having a particle size distribution D₉₀ value of about 25 μm or less, D₅₀ value of about 15 μm or less, and D₁₀ value between about 0.1 μm and 5 μm.

According to an eighth embodiment of above aspect, the sustained release oral pharmaceutical composition is an osmotic tablet, wherein the osmotic tablet is in the form of a single core osmotic tablet or a bilayer osmotic tablet.

According to a first embodiment of the eighth embodiment above, the single core osmotic tablet comprises:
(i) a core comprising tofacitinib, a diluent, a binder, optionally an acidifying agent, optionally a surfactant, and other pharmaceutically acceptable excipients;
(ii) a first coating over the core, wherein the first coating comprises a release controlling polymer and a coating additive; and
(iii) an outer modified release coating over the first coating, wherein the outer modified release coating comprises a modified release polymer and a coating additive.

According to a second embodiment of the eighth embodiment, the bilayer osmotic tablet comprises:
(i) a core comprising
   a. a drug layer comprising tofacitinib, a diluent, a release controlling polymer, optionally an acidifying agent, optionally a surfactant, optionally an osmogen, and other pharmaceutically acceptable excipients; and
   b. a push layer comprising a diluent, a release controlling polymer, and an osmogen;
(ii) a first coating over the core, wherein the first coating comprises a release controlling polymer and a coating additive; and
(iii) an outer modified release coating over the first coating, wherein the outer modified release coating comprises a modified release polymer and a coating additive.

According to a ninth embodiment of above aspect, the sustained release oral pharmaceutical composition is a sustained release matrix tablet.

According to a first embodiment of the ninth embodiment, the sustained release matrix tablet comprises:
(i) a core comprising tofacitinib, a diluent, a release controlling polymer, optionally an acidifying agent, optionally a surfactant, and other pharmaceutically acceptable excipients; and
(ii) an outer modified release coating, wherein the outer modified release coating comprises a modified release polymer and a coating additive.

According to a second embodiment of the ninth embodiment, the sustained release matrix tablet comprises:
(i) a core comprising tofacitinib, a diluent, a release controlling polymer, optionally an acidifying agent, optionally a surfactant, and other pharmaceutically acceptable excipients;
(ii) a first coating over the core, wherein the first coating comprises a release controlling polymer, a binder, and a coating additive; and
(iii) an outer modified release coating over the first coating, wherein the outer modified release coating comprises a modified release polymer and a coating additive.

According to a tenth embodiment of the above aspect, the sustained release oral pharmaceutical composition is a sustained release reservoir tablet.

According to a first embodiment of the tenth embodiment, the sustained release reservoir tablet comprises:
(i) a core comprising tofacitinib, a diluent, a binder, optionally an acidifying agent, optionally a surfactant, and other pharmaceutically acceptable excipients; and
(ii) an outer coating over the core, wherein the outer coating comprises a release controlling polymer, optionally a modified release polymer, and a coating additive.

The term “pharmaceutical composition,” as used herein, may include tablets, capsules, granules, and the like.

The term “tofacitinib,” as used herein, refers to tofacitinib free base or pharmaceutically acceptable salts, in particular pharmaceutically acceptable acid addition salts, e.g., citrate, hydrochloride, hydrobromide, hydroiodide, nitrate, sulfate, bisulfate, phosphate, acetate, lactate, tartarate, succinate, malate, maleate, oxalate, fumarate, gluconate, saccharate, benzoate, methansulfonate, ethanesulfonate, benzenesulfonate, and the like. The preferred pharmaceutically acceptable salt is citrate salt.

The compositions of the present invention comprise an outer modified release coating. The modified release coating may comprise a modified release polymer. The modified release polymer may be a pH dependent polymer, such as those marketed under the brand name Eudragit®, or a copolymer of dimethylaminoethyl methacrylate, butyl methacrylate, and methyl methacrylate, such as those marketed under the brand names Eudragit® E PO, Eudragit® E 100, and Eudragit® E 12.5; a copolymer of methacrylic acid or methacrylic acid esters e.g., copolymer based on methacrylic acid and ethyl acrylate (Eudragit® L 100-55), copolymers based on methacrylic acid and methyl methacrylate (Eudragit® S 100). Preferably, Eudragit® E PO and Eudragit® L 100-55 are used.

The term “release controlling polymer,” as used herein, refers to polymers which control the release of a drug. Such polymers may be present in the core of a tablet which helps in releasing the drug by matrix erosion or in the coating over a core which helps in releasing the drug by osmotic pressure, or by diffusion of the drug through coating.

These polymers may be:

(i) water swellable or water soluble or erodible polymers; or

(ii) water insoluble or non-erodible polymers.

In a matrix core, water swellable or water soluble or erodible polymers are either swellable or dissolvable or erodible in pure water or requiring the presence of an acid or base to ionize the polymeric matrix sufficiently to cause erosion or dissolution. When contacted with an aqueous environment, the polymer imbibes water and forms an aqueous-swollen gel or matrix that entraps tofacitinib. The aqueous swollen matrix gradually erodes, swells, disintegrates, disperses, or dissolves in the environment of use, thereby controlling the release of tofacitinib.

The water swellable or water soluble or erodible polymers include polyethylene oxide, in particular polyethylene oxide water soluble resins (Polyox® WSR Coagulant and Polyox® WSR-303); glyceryl fatty acid esters, e.g., glyceryl behenate, glyceryl monostearate, glycerol distearate, glycerol monooleate, acetylated monoglycerides, tristearin, tripalmitin, cetyl esters wax, glyceryl palmitostearate, and glyceryl behenate; hydrogenated castor oil; cellulose derivatives, e.g., hydroxyethyl cellulose, hydroxypropyl cellulose, hydroxypropyl methylcellulose, ethylhydroxy ethylcellulose, methylethyl cellulose, carboxymethyl cellulose, and carboxymethyl ethylcellulose; pullulan; polyvinyl pyrrolidone; polyvinyl alcohol; and polyvinyl acetate. Preferably, the water swellable or water soluble or erodible polymers include polyethylene oxide water soluble resins, glyceryl behenate, hydroxyethyl cellulose, hydroxypropyl methylcellulose, and polyvinyl pyrrolidone.

Water insoluble or non-erodible polymers control the drug release by osmotic pressure or by diffusion. Suitable water insoluble or non-erodible polymers include copolymers of methacrylic acid or methacrylic acid esters; polyvinyl chloride; polyethylene; cellulose and cellulose derivatives, e.g., ethylcellulose, cellulose acetate, cellulose acetate phthalate (CAP), hydroxypropyl methylcellulose acetate succinate (HPMCAS), hydroxypropyl methylcellulose phthalate (HPMCP), and cellulose acetate succinate (CAS); polyvinyl polymers, e.g., polyvinyl alcohol phthalate, polyvinyl acetate phthalate, and polyvinyl butyl phthalate; polyvinyl acetate or polyvinyl acetate copolymers; crosslinked polyvinylpyrrolidone (also known as crospovidone); and fatty compounds, e.g., carnauba wax, microcrystalline wax, and triglycerides; and mixtures of one or more of these polymers.

The term “particle size distribution” as used herein is defined by one or more of D₉₀ value, D₅₀ value, or D₁₀ value.

D₉₀ value is defined as the particle diameter at which 90% of the particles have a diameter less than the diameter which corresponds to that D₉₀ value. The D₉₀ value of tofacitinib particles in the present invention is about 30 μm or less, in particular about 25 μm or less, in particular between about 15 μm and 25 μm.

D₅₀ value, also known as median particle size, is defined as the particle diameter at which 50% of the particles have a diameter less than the diameter which corresponds to that D₅₀ value. The D₅₀ value of tofacitinib particles in the present invention is about 20 μm or less, in particular about 10 μm or less, in particular between about 1 μm and 10 μm.

D₁₀ value is defined as the particle diameter at which 10% of the particles have a diameter less than the diameter which corresponds to that D₁₀ value. The D₁₀ value of tofacitinib particles in the present invention is about 5 μm or less, in particular between about 0.1 μm and 5 μm.

The term “pharmaceutically acceptable excipients,” as used herein, includes any physiologically inert additives that are routinely used in pharmaceutical dosage forms. Pharmaceutically acceptable excipients are selected from the group comprising diluents, binders, osmogens, acidifying agents, surfactants, disintegrants, lubricants, and glidants.

Suitable diluents are selected from the group comprising lactose, e.g., directly compressible lactose (Pharmatose® DCL11), lactose monohydrate, lactose anhydrous, and spray dried lactose; microcrystalline cellulose, e.g., microcrystalline PH 112, microcrystalline PH 101, and microcrystalline PH 102; sugar alcohols, e.g., sorbitol, erythritol, xylitol, and mannitol; sugars, e.g., sucrose, DiPac® (a directly compressible, co-crystallized sugar consisting of 97% sucrose and 3% maltodextrin), and starch, e.g., pregelatinized starch. Preferably, the diluents are directly compressible lactose (preferably Pharmatose® DCL11), microcrystalline cellulose, sorbitol, pregelatinized starch, and combinations thereof.

Water soluble diluents, e.g., directly compressible lactose and sorbitol, may also act as an osmotic agent.

Suitable binders are selected from the group comprising povidone, in particular PVP K30; copovidone; celluloses, e.g., hydroxypropyl methylcellulose, in particular, HPMC E-5; hydroxy ethylcellulose, hydroxypropyl cellulose, methylcellulose, and ethylcellulose; starch, e.g., pregelatinized starch and low density starch; microcrystalline cellulose; lactose; xanthan gum; gum acacia; sodium alginate; propylene glycol; polyvinyl alcohol; corn syrup; methacrylates; carboxyvinyl polymers, e.g., carbomers; and combinations thereof.

The term “osmogens” as used herein, are water-soluble compounds capable of imbibing water and thereby establishing an osmotic pressure gradient across the barrier of the surrounding coating. Suitable osmogens are selected from the group comprising salts, e.g., sodium chloride, magnesium chloride, calcium chloride, potassium chloride, magnesium sulfate, potassium sulfate, sodium carbonate, and sodium sulfate; organic acids, e.g., ascorbic acid, benzoic acid, fumaric acid, and citric acid; and sugars, e.g., mannitol, sucrose, sorbitol, xylitol, lactose, dextrose, and trehalose.

The term “acidifying agents,” as used herein, are acidic substances which help in solubilizing the drug by creating an acidic microenvironment. Suitable acidifying agents are organic acids selected from the group comprising citric acid, fumaric acid, tartaric acid, succinic acid, malic acid, glutamic acid, and aspartic acid hydrates and acid salts thereof. Preferably, suitable acids are citric acid, fumaric acid, tartaric acid, and succinic acid. The acids may be used alone or in combinations thereof.

Suitable surfactants are selected from the group comprising polyoxyethylene sorbitan monooleate (Tween® 80), sodium lauryl sulphate, sorbitan monolaurate, sorbitan trioleate, polyoxyethylene sorbital, sorbitan tristearate, polyoxyethylene sorbital hexastearate, ethylene glycol fatty acid esters, propylene glycol fatty acid esters, propylene glycol monostearate, glycerol monostearate, and sorbitan monooleate. Preferably, suitable surfactants are polyoxyethylene sorbitan monooleate (Tween® 80) and sodium lauryl sulphate.

Suitable disintegrants are selected from the group comprising croscarmellose sodium, hydroxypropyl cellulose (L-HPC), crospovidone, carboxymethyl cellulose sodium, carboxymethyl cellulose calcium, sodium starch glycolate, gums, alginic acid or alginates, pregelatinized starch, corn starch, modified starch, carboxymethyl starch, polyacrylates, and combinations thereof.

Suitable glidants are selected from the group comprising magnesium stearate, stearic acid, calcium stearate, colloidal silicon dioxide, starch, talc, and combinations thereof.

Suitable lubricants are selected from the group comprising magnesium stearate, talc, and silica.

The term “osmotic tablet,” as used herein, includes an osmotic release oral system in the form of a tablet as known in the art. The osmotic tablet comprises a semipermeable membrane which may optionally comprise one or more laser drilled openings. The tablets can also be drilled manually. The term “semipermeable,” as used herein, includes a membrane through which water readily diffuses through the means of a membrane, but solutes dissolved in water typically cannot readily diffuse through the membrane. As the tablet passes through the body, the osmotic pressure of water entering the tablet pushes the drug through the opening(s) in the semipermeable membrane.

The term “single core osmotic tablet,” as used herein, includes an osmotic delivery system as known in the art. The single core osmotic tablets comprise a compressed core containing an osmotically effective composition surrounded by a semipermeable membrane.

The term “bilayer osmotic tablet,” as used herein, includes an osmotic delivery system in the form of a tablet comprising two adjacent layers, (i) a drug layer comprising the drug and a water soluble polymer, and (ii) a push layer or water swelling layer which comprises water swellable polymers and/or osmogens. The push layer does not contain the drug. The bilayer tablet is surrounded by a semipermeable membrane which comprises one or more laser drilled openings.

The term “sustained release matrix tablet,” as used herein, refers to a sustained release tablet which has a matrix system comprising of a release controlling polymer which sustains tofacitinib release.

The term “sustained release reservoir tablet,” as used herein, refers to a sustained release tablet comprising a coating of release controlling polymer imposed between a tofacitinib core and the elution medium. Drug release results from diffusion of the drug through the release controlling layer, permeation of the release controlling layer by water and/or erosion of the coating.

Suitable coating additives are selected from the group comprising pore formers, surfactants, plasticizers, anti-foaming agents, opacifiers, anti-tacking agents, coloring agents, coating solvents, and combinations thereof.

The term “pore former,” as used herein, may include the materials incorporated in a pharmaceutical composition, particularly in the coating or in the matrix, for forming a micro-porous membrane or micro-porous matrix. The micro-porous membrane or micro-porous matrix may be formed in situ by a pore former by its leaching during the operation of the system. Suitable pore formers are selected from the group comprising water soluble polymers, e.g., hydroxypropyl methylcellulose (HPMC); hydroxyethyl cellulose (HEC); hydroxypropyl cellulose (HPC); polyvinyl pyrrolidone; polyvinyl alcohol; polyvinyl acetate; surfactant, e.g., sorbitan monolaurate, sorbitan trioleate, polyoxyethylene sorbital, sorbitan tristearate, polyoxyethylene sorbital hexastearate, ethylene glycol fatty acid ester, propylene glycol fatty acid ester, propylene glycol monostearate, glycerol monostearate, and sorbitan monooleate; alkaline metal salts, e.g., sodium chloride, sodium bromide, and potassium chloride; alkaline earth metals, e.g., calcium chloride and calcium nitrate; carbohydrates, e.g., sucrose, glucose, fructose, mannose, lactose, sorbitol, and mannitol; and diols and polyols.

Suitable surfactants are selected from the group comprising sorbitan monolaurate, sorbitan trioleate, polyoxyethylene sorbital, sorbitan tristearate, polyoxyethylene sorbital hexastearate, ethylene glycol fatty acid ester, propylene glycol fatty acid ester, propylene glycol monostearate, glycerol monostearate, sorbitan monooleate, and combinations thereof.

Suitable plasticizers are selected from the group comprising triethyl citrate, dibutyl sebacate, acetylated triacetin, tributyl citrate, glyceryl tributyrate, monoglyceride, rapeseed oil, olive oil, sesame oil, acetyl tributyl citrate, acetyl triethyl citrate, glycerin, sorbitol, diethyloxalate, diethyl phthalate, diethyl malate, diethyl fumarate, dibutyl succinate, diethyl malonate, dioctyl phthalate, and combinations thereof.

A suitable anti-foaming agent is simethicone. Simethicone imparts smoothness to the coating.

Suitable opacifiers are selected from the group comprising titanium dioxide, manganese dioxide, iron oxide, silicon dioxide, and combinations thereof.

Suitable anti-tacking agents are selected from the group comprising talc, magnesium stearate, calcium stearate, stearic acid, silica, glyceryl monostearate, and combinations thereof.

Suitable coloring agents are selected from the group consisting of FD&C (Federal Food, Drug and Cosmetic Act) approved coloring agents; natural coloring agents; pigments, e.g. iron oxide, titanium dioxide, and zinc oxide; and combinations thereof.

Suitable coating solvents used for forming a solution or suspension for coating are selected from the group comprising water, ethanol, methylene chloride, isopropyl alcohol, acetone, methanol, and combinations thereof.

The “in-vitro release profile” of the pharmaceutical compositions of the present invention is the release profile obtained when dissolution testing was performed for tablets in USP type 2 apparatus at 50 r.p.m. in (i) pH 6.8 phosphate buffer/900 mL or (ii) 0.1N HCl/900 mL at 37° C.

The invention is further illustrated by the following examples, which are for illustrative purposes only and should not be construed as limiting the scope of the invention in anyway.

### EXAMPLES

### Example 1

### Osmotic Tablet

### Example 1(a)

| | |
|---|---|
| Ingredients | Quantity (mg/tablet) |

Core

| | |
|---|---|
| Tofacitinib citrate | 17.76 |
| Lactose | 152.24 |
| Hydroxypropyl methylcellulose | 16.00 |
| Polyvinylpyrrolidone | 12.00 |
| Magnesium stearate | 2.00 |
| Core tablet weight | 200.00 |

First coating (Extended release)

| | |
|---|---|
| Cellulose acetate | 9.60 |
| Polyethylene glycol | 6.40 |
| Acetone | q.s. |
| Purified water | q.s. |
| Coated tablet weight | 216.00 |

Laser drilled hole or without a hole
Outer coating

| | |
|---|---|
| Eudragit ® E PO | 5.71 |
| Sodium lauryl sulphate | 0.57 |
| Stearic acid | 0.86 |
| Talc | 2.86 |
| Purified water | q.s. |
| Total tablet weight | 226.00 |

### Procedure:

1. Tofacitinib citrate, hydroxypropyl methylcellulose, and polyvinylpyrrolidone were sifted successively.
2. Lactose was sifted.
3. The materials of step 1 and step 2 were mixed geometrically and blended in a V-blender for 20 minutes.
4. Magnesium stearate was sifted and added to the mixture of step 3, and then the mixture was blended in a V-blender for 20 minutes.
5. The mixture of step 4 was compressed into core tablets.
6. Cellulose acetate was dissolved in acetone; and polyethylene glycol was dissolved in purified water; and both solutions were mixed together under stirring.
7. The core tablets of step 5 were coated with the solution of step 6.
8. The tablets of step 7 were dried at 45° C. for 24 hours.
9. Optionally, a hole of approximately 1000 microns was made by laser ablation on the tablets of step 8.
10. Sodium lauryl sulfate, stearic acid, and Eudragit® E PO were dissolved in water.
11. Talc was added to the solution of step 10, and then the mixture was stirred for 15 minutes, and the dispersion was filtered.
12. The tablets of step 8 or step 9 were coated using the dispersion of step 11.
13. The tablets of step 12 were dried in a coating pan at 40° C. for 2 hours.

### Example 1(b)

| | |
|---|---|
| Ingredients | Quantity (mg/tablet) |

Core

| | |
|---|---|
| Tofacitinib citrate | 17.76 |
| Lactose | 132.24 |
| Citric Acid | 20.00 |
| Hydroxypropyl methylcellulose | 16.00 |
| Polyvinylpyrrolidone | 12.00 |
| Magnesium stearate | 2.00 |
| Core tablet weight | 200.00 |

First coating (Extended release)

| | |
|---|---|
| Cellulose acetate | 9.60 |
| Polyethylene glycol | 6.40 |
| Acetone | q.s. |
| Purified water | q.s. |
| Coated tablet weight | 216.00 |

Laser drilled hole or Without a hole
Outer coating

| | |
|---|---|
| Eudragit ® EPO | 5.71 |
| Sodium lauryl sulphate | 0.57 |
| Stearic acid | 0.86 |
| Talc | 2.86 |
| Purified water | q.s. |
| Total tablet weight | 226.00 |

### Procedure:

1. Tofacitinib citrate, hydroxypropyl methylcellulose, and polyvinylpyrrolidone were sifted successively.
2. Lactose and citric acid were sifted.
3. The materials of step 1 and step 2 were mixed geometrically and blended in a V-blender for 20 minutes.
4. Magnesium stearate was sifted and added to the mixture of step 3, and then the mixture was blended in a V-blender for 20 minutes.
5. The mixture of step 4 was compressed into core tablets.
6. Cellulose acetate was dissolved in acetone; and polyethylene glycol was dissolved in purified water; and both solutions were mixed together under stirring.
7. The core tablets of step 5 were coated with the solution of step 6.
8. The tablets of step 7 were dried at 45° C. for 24 hours.
9. Optionally, a hole of approximately 1000 microns was made by laser ablation on the tablets of step 8.
10. Sodium lauryl sulfate, stearic acid, and Eudragit® E PO were dissolved in water.
11. Talc was added to the solution of step 10 and the mixture was stirred for 15 minutes, and the dispersion was filtered.
12. The tablets of step 8 or step 9 were coated using the dispersion of step 11.
13. The tablets of step 12 were dried in a coating pan at 40° C. for 2 hours.

### Example 1(c)

| | |
|---|---|
| Ingredients | Quantity (mg/tablet) |

Core

| | |
|---|---|
| Tofacitinib citrate | 17.76 |
| Lactose | 142.24 |
| Sodium lauryl sulfate | 10.00 |
| Hydroxypropyl methylcellulose | 16.00 |
| Polyvinylpyrrolidone | 12.00 |
| Magnesium stearate | 2.00 |
| Core tablet weight | 200.00 |

First coating (Extended release)

| | |
|---|---|
| Cellulose acetate | 9.60 |
| Polyethylene glycol | 6.40 |
| Acetone | q.s. |
| Purified water | q.s. |
| Coated tablet weight | 216.00 |

Laser drilled hole or Without a hole
Outer coating

| | |
|---|---|
| Eudragit ® E PO | 5.71 |
| Sodium lauryl sulphate | 0.57 |
| Stearic acid | 0.86 |
| Talc | 2.86 |
| Purified water | q.s. |
| Total tablet weight | 226.00 |

### Procedure:

1. Tofacitinib citrate, hydroxypropyl methylcellulose, and polyvinylpyrrolidone were sifted successively.
2. Lactose and sodium lauryl sulfate were sifted.
3. The materials of step 1 and step 2 were mixed geometrically and blended in a V-blender for 20 minutes.
4. Magnesium stearate was sifted and added to the mixture of step 3, and then the mixture was blended in a V-blender for 20 minutes.
5. The mixture of step 4 was compressed into core tablets.
6. Cellulose acetate was dissolved in acetone; polyethylene glycol was dissolved in purified water; and both solutions were mixed together under stirring.
7. The core tablets of step 5 were coated with the solution of step 6.
8. The tablets of step 7 were dried at 45° C. for 24 hours.
9. Optionally, a hole of approximately 1000 microns was made by laser ablation on the tablets of step 8.
10. Sodium lauryl sulfate, stearic acid, and Eudragit® E PO were dissolved in water.
11. Talc was added to the solution of step 10 and the mixture was stirred for 15 minutes, and the dispersion was filtered.
12. The tablets of step 8 or step 9 were coated using the dispersion of step 11.
13. The tablets of step 12 were dried in a coating pan at 40° C. for 2 hours.

### Example 1(d)

| | |
|---|---|
| Ingredients | Quantity (mg/tablet) |

Core

| | |
|---|---|
| Tofacitinib citrate | 17.76 |
| Sorbitol | 152.24 |
| Hydroxy ethylcellulose | 16.00 |
| Copovidone | 12.00 |
| Magnesium stearate | 2.00 |
| Core tablet weight | 200.00 |

First coating (Extended release)

| | |
|---|---|
| Ethylcellulose | 9.60 |
| Hydroxypropyl methylcellulose | 6.40 |
| Isopropyl alcohol | q.s. |
| Purified water | q.s. |
| Coated tablet weight | 216.00 |

Without a drilled hole
Outer coating

| | |
|---|---|
| Eudragit ® E PO | 8.64 |
| Talc | 1.62 |
| Stearic acid | 0.54 |
| Purified water | q.s. |
| Total tablet weight | 226.80 |

### Procedure:

1. Tofacitinib citrate, hydroxy ethylcellulose, and copovidone were sifted successively.
2. Sorbitol was sifted.
3. The materials of step 1 and step 2 were mixed geometrically and blended in a V-blender for 20 minutes.
4. Magnesium stearate was sifted and added to the mixture of step 3, and then the mixture was blended in a V-blender for 20 minutes.
5. The mixture of step 4 was compressed into core tablets.
6. Ethylcellulose was dissolved in isopropyl alcohol, and then hydroxypropyl methylcellulose was added to the solution, followed by addition of purified water and stirring.
7. The core tablets of step 5 were coated with the dispersion of step 6.
8. The tablets of step 7 were dried at 45° C. for 2 hours.
9. Stearic acid and Eudragit® E PO were dissolved in water.
10. Talc was added to the solution of step 9 and then the mixture was stirred for 15 minutes, and the dispersion was filtered.
11. The tablets of step 8 were coated using the dispersion of step 10.
12. The tablets of step 11 were dried in a coating pan at 40° C. for 2 hours.

### Example 1(e)

| | |
|---|---|
| Ingredients | Quantity (mg/tablet) |

Core

| | |
|---|---|
| Tofacitinib citrate | 11.544 |
| Lactose | 158.460 |
| Hydroxy ethylcellulose | 16.000 |
| Copovidone | 12.000 |
| Magnesium stearate | 2.000 |
| Core tablet weight | 200.000 |

First coating (Extended release)

| | |
|---|---|
| Cellulose acetate | 8.400 |
| Polyethylene glycol | 5.600 |
| Acetone | q.s. |
| Purified water | q.s. |
| Coated tablet weight | 214.000 |

With or without a drilled hole
Outer coating

| | |
|---|---|
| Tofacitinib citrate | 6.216 |
| Eudragit ® E PO | 17.802 |
| Talc | 1.978 |
| Purified water | q.s. |
| Total tablet weight | 240.000 |

### Procedure:

1. Tofacitinib citrate, hydroxy ethylcellulose, and copovidone were sifted successively.
2. Lactose was sifted.
3. The materials of step 1 and step 2 were mixed geometrically and blended in a V-blender for 20 minutes.
4. Magnesium stearate was sifted, and then added to the mixture of step 3 and the resulting mixture was blended in a V-blender for 20 minutes.
5. The mixture of step 4 was compressed into core tablets.
6. Cellulose acetate was dissolved in acetone; polyethylene glycol was dissolved in purified water; and both solutions were mixed together.
7. The core tablets of step 5 were coated with the dispersion of step 6.
8. The tablets of step 7 were dried at 45° C. for 24 hours.
9. Optionally, a hole of approximately 500-1000 microns was made by laser ablation on the tablets of step 8.

10. Tofacitinib citrate and talc and were added into water and homogenized. Eudragit® E PO was successively added to the dispersion under stirring, and then stirred for 1 hour.

11. The tablets of step 8 or step 9 were coated using the dispersion of step 10.

12. The tablets of step 11 were dried in a coating pan at 40° C. for 2 hours.

### Example 1(f)

| | |
|---|---|
| Ingredients | Quantity (mg/tablet) |

Core
Drug Layer

| | |
|---|---|
| Tofacitinib citrate | 17.77 |
| Polyethylene oxide water soluble resin | 130.47 |
| Sodium chloride | 50.10 |
| Magnesium stearate | 1.67 |
| Drug layer weight | 200.00 |

Push Layer

| | |
|---|---|
| Polyethylene oxide water soluble resin | 61.98 |
| Microcrystalline cellulose | 16.60 |
| Sodium chloride | 20.75 |
| Iron oxide red | 0.25 |
| Magnesium stearate | 0.42 |
| Push layer weight | 100.00 |

First coating (Extended release)

| | |
|---|---|
| Cellulose acetate | 28.50 |
| Polyethylene glycol | 1.50 |
| Acetone | q.s. |
| Purified Water | q.s. |
| Coated tablet weight | 330.00 |

Outer coating

| | |
|---|---|
| Eudragit ® E PO | 16.00 |
| Polyethylene glycol | 1.60 |
| Talc | 2.40 |
| Isopropyl alcohol | q.s. |
| Acetone | q.s. |
| Total tablet weight | 350.00 |

### Procedure:

Drug Layer
1. Tofacitinib citrate was sifted, followed by sodium chloride and polyethylene oxide water soluble resin.
2. The sieved materials of step 1 were blended in a V-blender.
3. Magnesium stearate was sifted, and then added to the blend of step 2 and mixed.

Push Layer
4. Polyethylene oxide water soluble resin, microcrystalline cellulose, and sodium chloride were sifted.
5. Magnesium stearate and iron oxide red were sifted.
6. The materials of steps 4 and 5 were blended in a V-blender.

Compression
7. The materials of step 3 and 6 were compressed to form a bilayer tablet using a suitable bilayer press.

Coating
8. Cellulose acetate and polyethylene glycol were dissolved in water and mixed under stirring.
9. The tablets of step 7 were coated using the solution of step 8.
10. The coated tablets of step 9 were dried.
11. A hole of approximately 5000 microns was made by laser ablation on the tablets of step 10.
12. Eudragit® E PO and polyethylene glycol were dissolved in an acetone-isopropyl alcohol mixture, and then talc was added to the mixture.
13. The dispersion of step 12 was filtered.
14. The tablets of step 11 were coated using the dispersion of step 13.
15. The coated tablets of step 14 were dried.

### Example 1(g)

| | |
|---|---|
| Ingredients | Quantity (mg/tablet) |

Core
Drug Layer

| | |
|---|---|
| Tofacitinib citrate | 17.77 |
| Polyethylene oxide water soluble resin | 180.56 |
| Magnesium stearate | 1.67 |
| Drug layer weight | 200.00 |

Push Layer

| | |
|---|---|
| Polyethylene oxide water soluble resin | 78.58 |
| Sodium chloride | 20.75 |
| Iron oxide red | 0.25 |
| Magnesium stearate | 0.42 |
| Push layer weight | 100.00 |

First coating (Extended release)

| | |
|---|---|
| Cellulose acetate | 28.50 |
| Polyethylene glycol | 1.50 |
| Acetone | q.s. |
| Purified water | q.s. |
| Coated tablet weight | 330.00 |

Outer coating

| | |
|---|---|
| Eudragit ® E PO | 16.00 |
| Polyethylene glycol | 1.60 |
| Talc | 2.40 |
| Isopropyl alcohol | q.s. |
| Acetone | q.s. |
| Total tablet weight | 350.00 |

### Procedure:

Drug Layer
1. Tofacitinib citrate was sifted, followed by polyethylene oxide water soluble resin.
2. The sieved materials of step 1 were blended in a V-blender.
3. Magnesium stearate was sifted, and then added to the blend of step 2 and mixed.

Push Layer
4. Polyethylene oxide water soluble resin and sodium chloride were sifted.
5. Magnesium stearate and iron oxide red were sifted.
6. The materials of step 5 and 6 were blended in a V-blender.

Compression
7. The materials of step 3 and 6 were compressed to form a bilayer tablet using a suitable bilayer press.

Coating
8. Cellulose acetate and polyethylene glycol were dissolved in water.
9. The tablets of step 7 were coated using the solution of step 8.
10. The coated tablets of step 9 were dried.
11. A hole of approximately 5000 microns was made by laser ablation on the tablets of step 10.
12. Eudragit® E PO and polyethylene glycol were dissolved in an acetone-isopropyl alcohol mixture, and then talc was added to the mixture.
13. The dispersion of step 12 was filtered.
14. The tablets of step 11 were coated using the dispersion of step 13.
15. The coated tablets of step 14 were dried.

### Example 1(h)

| | |
|---|---|
| Ingredients | Quantity (mg/tablet) |

Core
Drug Layer

| | |
|---|---|
| Tofacitinib citrate | 17.764 |
| Polyethylene oxide water soluble resin | 89.236 |
| Sodium chloride | 46.000 |
| Magnesium stearate | 1.000 |
| Drug layer weight | 154.000 |

Push Layer

| | |
|---|---|
| Polyethylene oxide water soluble resin | 48.760 |
| Sodium chloride | 21.470 |
| Iron oxide red | 0.350 |
| Magnesium stearate | 0.420 |
| Push layer weight | 71.000 |
| Tablet core weight | 225.000 |

First coating (Extended release)

| | |
|---|---|
| Cellulose acetate | 27.920 |
| Polyethylene glycol | 10.320 |
| Acetone | q.s. |
| Purified water | q.s. |
| Coated tablet weight | 263.250 |

Outer coating

| | |
|---|---|
| Eudragit ® E PO | 6.310 |
| Triethyl citrate | 0.630 |
| Talc | 0.940 |
| Acetone | qs |
| Purified Water | qs |
| Total tablet weight | 271.150 |

### Procedure:

Drug Layer
1. Tofacitinib citrate was sifted along with sodium chloride.
2. The material of step 1 was co-sifted with polyethylene oxide water soluble resin and the mixture was blended.
3. Magnesium stearate was sifted, and then added to the blend of step 2 and mixed.

Push Layer
4. Sodium chloride and polyethylene oxide water soluble resin were sifted.
5. Magnesium stearate and iron oxide red were sifted.
6. The materials of steps 4 and 5 were blended in a V-blender.

Compression
7. The materials of step 3 and 6 were compressed to form a bilayer tablet using a suitable bilayer press.

Coating
8. Cellulose acetate was added to acetone.
9. Polyethylene glycol was dissolved in purified water; this solution was added to the solution of step 8.
10. The tablets of step 7 were coated with the solution of step 9, and then dried.
11. A hole of approximately 5000 microns was made by laser ablation on the tablets of step 10.
12. Eudragit® E PO was dissolved in acetone, followed by the addition of triethyl citrate, talc, and purified water.
13. The tablets of step 11 were coated using the dispersion of step 12.

### Example 1(i)

| | |
|---|---|
| Ingredients | Quantity (mg/tablet) |

Core
Drug Layer

| | |
|---|---|
| Tofacitinib citrate | 17.760 |
| Polyethylene oxide water soluble resin | 87.230 |
| Sodium chloride | 46.000 |
| Magnesium stearate | 2.000 |
| Colloidal silicon dioxide | 1.000 |
| Drug Layer weight | 154.000 |

Push Layer

| | |
|---|---|
| Polyethylene oxide water soluble resin | 48.760 |
| Sodium chloride | 21.470 |
| Ferric oxide | 0.350 |
| Magnesium stearate | 0.420 |
| Push Layer Weight | 71.000 |
| Bilayer Tablet weight | 225.000 |

First coating (Extended release)

| | |
|---|---|
| Cellulose acetate | 11.498 |
| Polyethylene glycol | 4.253 |
| Acetone | q.s. |
| Purified water | q.s. |
| Coated tablet weight | 240.751 |

Outer coating

| | |
|---|---|
| Eudragit ® E PO | 5.695 |
| Polyethylene glycol | 0.570 |
| Talc | 0.833 |
| Titanium dioxide | 2.058 |
| Ferric oxide | 0.093 |
| Acetone | q.s. |
| Purified water | q.s. |
| Total tablet weight | 250.000 |

### Procedure:

Drug Layer
1. Tofacitinib citrate was sifted with half of the polyethylene oxide water soluble resin, and retentions were collected separately.
2. The material of step 1 was blended.
3. The remaining polyethylene oxide water soluble resin was sifted, and retentions were collected separately.
4. The sifted material of step 3 was blended with the blend of step 2.
5. The oversize materials of step 1 and step 3 were sifted with sodium chloride.
6. The materials of step 4 and step 5 were blended.
7. Magnesium stearate and colloidal silicon dioxide were sifted, and then added to the blend of step 6 and mixed.

Push Layer
8. Ferric oxide and a part of polyethylene oxide water soluble resin were sifted.
9. Sodium chloride, the retained material of step 8, and the remaining part of polyethylene oxide water soluble resin were sifted.
10. The materials of step 8 and step 9 were blended.
11. Magnesium stearate was sifted.
12. The blend of step 10 was lubricated with the sifted magnesium stearate of step 11.

Compression
13. The materials of step 7 and step 12 were compressed to form a bilayer tablet using a suitable bilayer press.

Coating
14. Cellulose acetate was added to acetone.
15. Polyethylene glycol was dissolved in purified water, and the solution was added to the solution of step 14.
16. The tablets of step 13 were coated with the solution of step 15 and then dried.
17. The tablets of step 16 were drilled with an orifice.
18. Eudragit® E PO was dissolved in acetone.
19. Polyethylene glycol was dissolved in purified water and the solution was added to the solution of step 18.
20. Talc was added to the solution of step 19.
21. Ferric oxide and titanium dioxide were homogenized in purified water and the resulting solution was added to the solution of step 20.
22. The tablets of step 17 were coated with the solution of step 21.

### Example 2

### Matrix Tablet

### Example 2(a)

| | |
|---|---|
| Ingredients | Quantity (mg/tablet) |

Core

| | |
|---|---|
| Tofacitinib citrate | 17.76 |
| Polyethylene oxide water soluble resin | 150.24 |
| Microcrystalline cellulose | 30.00 |
| Magnesium stearate | 2.00 |
| Core tablet weight | 200.00 |

Outer coating

| | |
|---|---|
| Eudragit ® E PO | 11.43 |
| Sodium lauryl sulfate | 1.15 |
| Stearic acid | 1.71 |
| Talc | 5.71 |
| Purified water | q.s. |
| Total tablet weight | 220.00 |

### Procedure:

1. Tofacitinib citrate was sifted, followed by the sifting of microcrystalline cellulose.
2. Polyethylene oxide water soluble resin was sifted.
3. The materials of step 1 and step 2 were mixed geometrically and blended in a V-blender for 20 minutes.
4. Magnesium stearate was sifted, and then added to the mixture of step 3 and blended in a V-blender for 20 minutes.
5. The material of step 4 was compressed into core tablets.
6. Sodium lauryl sulfate, stearic acid, and Eudragit® E PO were dissolved in water.
7. Talc was added to the solution of step 6, and then stirred for 15 minutes. The dispersion so obtained was filtered.
8. The core tablets of step 5 were coated using the dispersion of step 7.
9. The tablets of step 8 were dried in a coating pan at 40° C. for 2 hours.

### Example 2(b)

| | |
|---|---|
| Ingredients | Quantity (mg/tablet) |

Core

| | |
|---|---|
| Tofacitinib citrate | 17.76 |
| Polyethylene oxide water soluble resin | 130.24 |
| Citric acid | 20.00 |
| Microcrystalline cellulose | 30.00 |
| Magnesium stearate | 2.00 |
| Core tablet weight | 200.00 |

Outer coating

| | |
|---|---|
| Eudragit ® E PO | 11.43 |
| Sodium lauryl sulfate | 1.15 |
| Stearic acid | 1.71 |
| Talc | 5.71 |
| Purified water | q.s. |
| Total tablet weight | 220.00 |

### Procedure:

1. Tofacitinib citrate was sifted, followed by sifting of microcrystalline cellulose and citric acid.
2. Polyethylene oxide water soluble resin was sifted.
3. The materials of step 1 and step 2 were mixed geometrically and blended in a V-blender for 20 minutes.
4. Magnesium stearate was sifted, and then added to the mixture of step 3 and blended in the V-blender for 20 minutes.
5. The material of step 4 was compressed into core tablets.
6. Sodium lauryl sulfate, stearic acid, and Eudragit® E PO were dissolved in water.
7. Talc was added to the solution of step 6 and the mixture was stirred for 15 minutes. The dispersion so obtained was filtered.
8. The core tablets of step 5 were coated using the dispersion of step 7.
9. The tablets of step 8 were dried in a coating pan at 40° C. for 2 hours.

### Example 2(c)

| | |
|---|---|
| Ingredients | Quantity (mg/tablet) |

Core

| | |
|---|---|
| Tofacitinib citrate | 17.76 |
| Polyethylene oxide water soluble resin | 140.24 |
| Sodium lauryl sulfate | 10.00 |
| Microcrystalline cellulose | 30.00 |
| Magnesium stearate | 2.00 |
| Core tablet weight | 200.00 |

Outer coating

| | |
|---|---|
| Eudragit ® E PO | 11.43 |
| Sodium lauryl sulfate | 1.15 |
| Stearic acid | 1.71 |
| Talc | 5.71 |
| Purified water | q.s. |
| Total tablet weight | 220.00 |

### Procedure:

1. Tofacitinib citrate was sifted, followed by sifting of microcrystalline cellulose and sodium lauryl sulfate.
2. Polyethylene oxide water soluble resin was sifted.
3. The materials of step 1 and step 2 were mixed geometrically and blended in a V-blender for 20 minutes.
4. Magnesium stearate was sifted, and then added to the mixture of step 3 and blended in the V-blender for 20 minutes.
5. The material of step 4 was compressed into core tablets.
6. Sodium lauryl sulfate, stearic acid, and Eudragit® E PO were dissolved in water.
7. Talc was added to the solution of step 6 and the mixture was stirred for 15 minutes. The dispersion so obtained was filtered.
8. The core tablets of step 5 were coated using the dispersion of step 7.
9. The tablets of step 8 were dried in a coating pan at 40° C. for 2 hours.

### Example 2(d)

| | |
|---|---|
| Ingredients | Quantity (mg/tablet) |

Core

| | |
|---|---|
| Tofacitinib citrate | 17.760 |
| Polyethylene oxide water soluble resin | 150.240 |
| Microcrystalline cellulose | 30.000 |
| Magnesium stearate | 2.000 |
| Core tablet weight | 200.00 |

First coating (Extended release)

| | |
|---|---|
| Ethylcellulose | 11.20 |
| Polyvinylpyrrolidone | 4.80 |
| Isopropyl alcohol | q.s. |
| Purified water | q.s. |
| Coated tablet weight | 216.00 |

Outer coating

| | |
|---|---|
| Eudragit ® E PO | 11.43 |
| Sodium lauryl sulfate | 1.15 |
| Stearic acid | 1.71 |
| Talc | 5.71 |
| Total tablet weight | 236.00 |

### Procedure:

1. Tofacitinib citrate was sifted, followed by sifting of microcrystalline cellulose.
2. Polyethylene oxide water soluble resin was sifted.
3. The materials of step 1 and step 2 were mixed geometrically and blended in a V-blender for 20 minutes.
4. Magnesium stearate was sifted, and then added to the mixture of step 3 and blended in the V-blender for 20 minutes.
5. The material of step 4 was compressed into core tablets.
6. Ethylcellulose and polyvinylpyrrolidone were dissolved in isopropyl alcohol; purified water was then added to the solution and the solution was stirred.
7. The core tablets of step 5 were coated using the dispersion of step 6.
8. The tablets of step 7 were dried in a coating pan at 40° C. for 2 hours.
9. Sodium lauryl sulfate, stearic acid, and Eudragit® E PO were dissolved in water.
10. Talc was added to the solution of step 9, and the mixture was stirred for 15 minutes. The dispersion so obtained was filtered.
11. The tablets of step 8 were coated using the dispersion of step 10.
12. The tablets of step 11 were dried in a coating pan at 40° C. for 2 hours.

### Example 2(e)

| | |
|---|---|
| Ingredients | Quantity (mg/tablet) |

Core

| | |
|---|---|
| Tofacitinib citrate | 17.76 |
| Glyceryl behenate | 150.24 |
| Microcrystalline cellulose | 30.00 |
| Magnesium stearate | 2.00 |
| Core tablet weight | 200.00 |

First coating (Extended release)

| | |
|---|---|
| Ethylcellulose | 8.00 |
| Polyvinylpyrrolidone | 8.00 |
| Isopropyl alcohol | q.s. |
| Purified water | q.s. |
| Outer coating | 216.00 |
| Eudragit ® E PO | 11.43 |
| Sodium lauryl sulfate | 1.15 |
| Stearic acid | 1.71 |
| Talc | 5.71 |
| Total tablet weight | 236.00 |

### Procedure:

1. Tofacitinib citrate was sifted, followed by sifting of microcrystalline cellulose.
2. Glyceryl behenate was sifted.
3. The materials of step 1 and step 2 were mixed geometrically and blended in a V-blender for 20 minutes.
4. Magnesium stearate was sifted, and then added to the mixture of step 3 and blended in a V-blender for 20 minutes.
5. The material of step 4 was compressed into core tablets.
6. Ethylcellulose and polyvinylpyrrolidone were dissolved in isopropyl alcohol; purified water was then added to the solution and the mixture was stirred.
7. The core tablets of step 5 were coated using the dispersion of step 6.
8. The tablets of step 7 were dried in a coating pan at 40° C. for 2 hours.
9. Sodium lauryl sulfate, stearic acid, and Eudragit® E PO were dissolved in water.
10. Talc was added to the solution of step 9, and the mixture was stirred for 15 minutes. The dispersion so obtained was filtered.
11. The tablets of step 8 were coated using the dispersion of step 10.
12. The tablets of step 11 were dried in a coating pan at 40° C. for 2 hours.

### Example 2(f)

| | |
|---|---|
| Ingredients | Quantity (mg/tablet) |

Core

| | |
|---|---|
| Tofacitinib citrate | 17.764 |
| Lactose monohydrate | 105.986 |
| Hydroxypropyl methylcellulose | 18.000 |
| Povidone | 18.000 |
| Fumaric acid | 63.000 |
| Magnesium stearate | 2.250 |
| Core tablet weight | 225.000 |

Outer coating

| | |
|---|---|
| Eudragit ® L 100-55 | 10.500 |
| Triethyl citrate | 0.900 |
| Talc | 3.600 |
| Isopropyl alcohol | q.s. |
| Total tablet weight | 238.500 |

### Procedure:

1. Tofacitinib citrate and fumaric acid were mixed together.
2. The material of step 1 was sifted.
3. Lactose monohydrate, hydroxypropyl methylcellulose, and povidone were sifted, and then blended with the material of step 2.
4. Magnesium stearate was sifted, and then added to the material of step 3.
5. The material of step 4 was compressed into core tablets.
6. Eudragit® L 100-55 was dissolved in isopropyl alcohol under stirring to obtain a clear solution.
7. Triethyl citrate was dispersed in the solution of step 6 with stirring for 10 minutes.
8. Talc was dispersed in the dispersion of step 7.
9. The core tablets of step 5 were coated using the dispersion of step 8.

### Example 3

### Reservoir Tablet

### Example 3(a)

| | |
|---|---|
| Ingredients | Quantity (mg/tablet) |

Core

| | |
|---|---|
| Tofacitinib citrate | 17.76 |
| Mannitol | 152.24 |
| Hydroxy ethylcellulose | 16.00 |
| Copovidone | 12.00 |
| Magnesium stearate | 2.00 |
| Core tablet weight | 200.00 |

Outer coating

| | |
|---|---|
| Ethylcellulose | 10.00 |
| Hydroxypropyl methylcellulose phthalate | 6.00 |
| Triethyl citrate | 2.00 |
| Talc | 2.00 |
| Acetone | q.s. |
| Purified water | q.s. |
| Total tablet weight | 220.00 |

### Procedure:

1. Tofacitinib citrate was sifted, followed by sifting of hydroxy ethylcellulose and copovidone.
2. Mannitol was sifted.
3. The materials of step 1 and step 2 were mixed geometrically and blended in a V-blender for 20 minutes.
4. Magnesium stearate was sifted, and then added to the mixture of step 3 and blended in the V-blender for 20 minutes.
5. The material of step 4 was compressed into core tablets.
6. Ethylcellulose, hydroxypropyl methylcellulose phthalate, and triethyl citrate were dissolved in acetone.
7. Talc was homogenized in purified water, and then added to the solution of step 6 and stirred for 45 minutes.
8. The core tablets of step 5 were coated with the solution of step 7.
9. The tablets of step 8 were dried in a coating pan for approximately 2 hours.

### Example 3(b)

| | |
|---|---|
| Ingredients | Quantity (mg/tablet) |

Core

| | |
|---|---|
| Tofacitinib citrate | 17.76 |
| Mannitol | 152.24 |
| Hydroxy ethylcellulose | 16.00 |
| Copovidone | 12.00 |
| Magnesium stearate | 2.00 |
| Core tablet weight | 200.00 |

Outer coating

| | |
|---|---|
| Ethylcellulose | 14.00 |
| Eudragit ® E 100 | 2.00 |
| Triethyl citrate | 2.00 |
| Talc | 2.00 |
| Acetone | q.s. |
| Purified water | q.s. |
| Total tablet weight | 220.00 |

### Procedure:

1. Tofacitinib citrate was sifted, sieved followed by sifting of hydroxy ethylcellulose and copovidone.
2. Mannitol was sifted.
3. The materials of step 1 and step 2 were mixed geometrically and blended in a V-blender for 20 minutes.
4. Magnesium stearate was sifted, and then added to the mixture of step 3 and blended in a V-blender for 20 minutes.
5. The material of step 4 was compressed into core tablets.
6. Ethylcellulose and Eudragit® E 100 were dissolved in triethyl citrate and acetone.
7. Talc was homogenized in purified water, and then added to the solution of step 6 and stirred for 45 minutes.
8. The core tablets of step 5 were coated with the solution of step 7.
9. The tablets of step 8 were dried in a coating pan for approximately 2 hours.

### Example 3(c)

| | |
|---|---|
| Ingredients | Quantity (mg/tablet) |

Core

| | |
|---|---|
| Tofacitinib citrate | 17.76 |
| Mannitol | 132.24 |
| Hydroxy ethylcellulose | 16.00 |
| Copovidone | 12.00 |
| Citric acid | 20.00 |
| Magnesium stearate | 2.00 |
| Core tablet weight | 200.00 |

Outer coating

| | |
|---|---|
| Ethylcellulose | 14.00 |
| Eudragit ® E 100 | 2.00 |
| Triethyl citrate | 2.00 |
| Talc | 2.00 |
| Acetone | q.s. |
| Purified water | q.s. |
| Total tablet weight | 220.00 |

### Procedure:

1. Tofacitinib citrate was sifted, followed by sifting of hydroxy ethylcellulose and copovidone.
2. Mannitol and citric acid were sifted.
3. The materials of step 1 and step 2 were mixed geometrically and blended in a V-blender for 20 minutes.
4. Magnesium stearate was sifted, and then added to the mixture of step 3 and blended in a V-blender for 20 minutes.
5. The material of step 4 was compressed into core tablets.
6. Ethylcellulose and Eudragit® E 100 were dissolved in triethyl citrate and acetone.
7. Talc was homogenized in purified water, and then added to the solution of step 6 and stirred for 45 minutes.
8. The core tablets of step 5 were coated with the solution of step 7.
9. The tablets of step 8 were dried in a coating pan for approximately 2 hours.

### Example 3(d)

| | |
|---|---|
| Ingredients | Quantity (mg/tablet) |

Core

| | |
|---|---|
| Tofacitinib citrate | 17.76 |
| Mannitol | 142.24 |
| Hydroxy ethylcellulose | 16.00 |
| Copovidone | 12.00 |
| Sodium lauryl sulfate | 10.00 |
| Magnesium stearate | 2.00 |
| Core tablet weight | 200.00 |

Outer coating

| | |
|---|---|
| Ethylcellulose | 14.00 |
| Eudragit ® E 100 | 2.00 |
| Triethyl citrate | 2.00 |
| Talc | 2.00 |
| Acetone | q.s. |
| Purified water | q.s. |
| Total tablet weight | 220.00 |

### Procedure:

1. Tofacitinib citrate was sifted, followed by sifting of hydroxy ethylcellulose and copovidone.
2. Mannitol and sodium lauryl sulfate were sifted.
3. The materials of step 1 and step 2 were mixed geometrically and blended in a V-blender for 20 minutes.
4. Magnesium stearate was sifted, and then added to the mixture of step 3 and blended in the V-blender for 20 minutes.
5. The material of step 4 was compressed into core tablets.
6. Ethylcellulose and Eudragit® E 100 were dissolved in triethyl citrate and acetone.
7. Talc was homogenized in purified water, and then added to the solution of step 6 and stirred for 45 minutes.
8. The core tablets of step 5 were coated with the solution of step 7.
9. The tablets of step 8 were dried in a coating pan for approximately 2 hours.

### Example 4

In-vitro dissolution testing was performed for tablets prepared according to Examples 1 to 2 in USP type 2 apparatus at 50 r.p.m. in pH 6.8 phosphate buffer/900 mL (Example 1(a), Example 1(f), Example 1(i), and Example 2(a)) and 0.1N HCl/900 mL (Example 2(a)) at 37° C. The results are presented in Table 1 below.

TABLE 1
In-vitro drug release

| | Percent Drug Release | | | | | |
|---|---|---|---|---|---|---|
| | Ex. 1(a) | Ex. 1(f) | Ex. 1(i) | Ex. 2(a) | | Ex. 2(f) |
| | pH 6.8 | pH 6.8 | pH 6.8 | pH 6.8 | Ex. 2(a) | pH 6.8 |
| Time | Phosphate | Phosphate | Phosphate | Phosphate | 0.1N | Phosphate |
| (hour) | buffer/900 mL | buffer/900 mL | buffer/900 mL | buffer/900 mL | HCl/900 mL | buffer/900 mL |
| 0.5 | — | — | — | — | — | 11 |
| 1 | 1 | 0 | 0 | 0 | 29 | 20 |
| 2.5 | 2 | 1 | 19 | 1 | 49 | 30 |
| 3 | — | — | — | — | — | 40 |
| 4 | — | — | — | — | — | 50 |
| 5 | 2 | 6 | 65 | 5 | 67 | 76 |
| 6 | 2 | 12 | — | 10 | 75 | 94 |
| 7 | — | 22 | — | 17 | — | 100 |
| 8 | 2 | — | — | 25 | 81 | 100 |

We claim:

1. A sustained release oral pharmaceutical composition of tofacitinib comprising tofacitinib, a release controlling polymer, and pharmaceutically acceptable excipients, wherein the sustained release oral pharmaceutical composition further comprises an outer modified release coating.

2. The sustained release oral pharmaceutical composition according to claim 1, wherein the composition comprises a core comprising tofacitinib, optionally a first coating over the core, and the outer modified release coating either over the core or over the first coating.

3. The sustained release oral pharmaceutical composition according to claim 2, wherein the first coating comprises from about 5% by weight to about 20% by weight of the core weight, and the outer modified release coating comprises from about 1% by weight to about 15% by weight either of the core weight or the weight of the coated core coated with the first coating.

4. The sustained release oral pharmaceutical composition according to claim 1, wherein the composition further comprises an acidifying agent or a surfactant or combinations thereof.

5. The sustained release oral pharmaceutical composition according to claim 1, wherein the composition has an in-vitro release profile such that the pharmaceutical composition releases not more than 30% of tofacitinib in 1 hour, not less than 35% and not more than 75% of tofacitinib in 2.5 hours, and not less than 75% of tofacitinib in 5 hours.

6. The sustained release oral pharmaceutical composition according to claim 1, wherein the composition has a release profile such that it releases less than 35% of tofacitinib in 2.5 hours.

7. The sustained release oral pharmaceutical composition according to claim 1, wherein the composition has a release profile such that it releases less than 75% of tofacitinib in 5 hours.

8. The sustained release oral pharmaceutical composition according to claim 1, wherein the composition further comprises an acidifying agent or a surfactant or combinations thereof.

9. The sustained release oral pharmaceutical composition according to claim 1, wherein the composition comprises tofacitinib having a particle size distribution D₉₀ value of about 30 μm or less, a D₅₀ value of about 20 μm or less, and a D₁₀ value of about 5 μm or less.

10. The sustained release oral pharmaceutical composition according to claim 9, wherein the composition comprises tofacitinib having a particle size distribution D₉₀ value of about 25 μm or less, a D₅₀ value of about 15 μm or less, and a D₁₀ value between about 0.1 μm and 5 μm.

11. The sustained release oral pharmaceutical composition according to claim 1, wherein the composition is an osmotic tablet, wherein the osmotic tablet is a single core osmotic tablet or a bilayer osmotic tablet.

12. The sustained release oral pharmaceutical composition according to claim 11, wherein the single core osmotic tablet comprises:
(i) a core comprising tofacitinib, a diluent, a binder, optionally an acidifying agent, optionally a surfactant, and other pharmaceutically acceptable excipients;
(ii) a first coating over the core, wherein the first coating comprises a release controlling polymer and a coating additive; and
(iii) the outer modified release coating over the first coating, wherein the outer modified release coating comprises a modified release polymer and a coating additive.

13. The sustained release oral pharmaceutical composition according to claim 11, wherein the bilayer osmotic tablet comprises:
a core comprising
a. a drug layer comprising tofacitinib, a diluent, a release controlling polymer, optionally an acidifying agent, optionally a surfactant, optionally an osmogen, and other pharmaceutically acceptable excipients; and
b. a push layer comprising a diluent, a release controlling polymer, and an osmogen;
(ii) a first coating over the core, wherein the first coating comprises a release controlling polymer and a coating additive; and
(iii) the outer modified release coating over the first coating, wherein the outer modified release coating comprises a modified release polymer and a coating additive.

14. The sustained release oral pharmaceutical composition according to claim 1, wherein the composition is a sustained release matrix tablet.

15. The sustained release oral pharmaceutical composition according to claim 14, wherein the sustained release matrix tablet comprises:
(i) a core comprising tofacitinib, a diluent, a release controlling polymer, optionally an acidifying agent, optionally a surfactant, and other pharmaceutically acceptable excipients; and
(ii) the outer modified release coating over the core, wherein the outer modified release coating comprises a modified release polymer and a coating additive.

16. The sustained release oral pharmaceutical composition according to claim 14, wherein the sustained release matrix tablet comprises:
(i) a core comprising tofacitinib, a diluent, a release controlling polymer, optionally an acidifying agent, optionally a surfactant, and other pharmaceutically acceptable excipients;
(ii) a first coating over the core, wherein the first coating comprises a release controlling polymer, a binder, and a coating additive; and
(iii) the outer modified release coating over the first coating, wherein the outer modified release coating comprises a modified release polymer and a coating additive.

17. The sustained release oral pharmaceutical composition according to claim 1, wherein the sustained release oral pharmaceutical composition is a sustained release reservoir tablet.

18. The sustained release oral pharmaceutical composition according to claim 17, wherein the sustained release reservoir tablet comprises:
(i) a core comprising tofacitinib, a diluent, a binder, optionally an acidifying agent, optionally a surfactant, and other pharmaceutically acceptable excipients; and
(ii) an outer coating over the core, wherein the outer coating comprises a release controlling polymer, optionally a modified release polymer and a coating additive.
